(19) United States
(12) Patent Application Publication
(10) Pub. No.: US 2026/0041694 A1
(43) Pub. Date: Feb. 12, 2026

(54) Udonitrectag For Topical Use In The Treatment Of Dermatological Diseases

(71) Applicants: MIMETECH S.R.L., Firenze (IT); MINERVA S.R.L., Firenze (IT)

(72) Inventors: Antonio GUARNA, Paola (IT); Giuseppe CASTRONOVO, Firenze (IT); Michele TANTURLI, Agliana (IT); Federico COZZOLINO, Fiesole (IT); Federica RONCHI, Milano (IT); Celestino RONCHI, Milano (IT); Caterina UGOLINI, Morciola di Vallefoglia (IT); Angelo COLOMBO, Brugherio (IT); Stefano RIZZA, Roma (IT)

(21) Appl. No.: 19/100,661

(22) PCT Filed: Aug. 3, 2023
(86) PCT No.: PCT/IB2023/057847

§ 371 Date: Feb. 3, 2025

(30) Foreign Application Priority Data
Aug. 4, 2022 (IT) 102022000016614

(51) Int. Cl. A61K 31/553; A61K 9/00; A61K 9/06; A61K 9/08; A61K 9/107; A61K 47/10; A61K 47/12; A61K 47/14; A61K 47/18; A61K 47/22; A61K 47/24; A61K 47/26; A61K 47/32; A61K 47/36; A61P 17/00; A61P 17/02; A61P 17/06; A61P 17/14

(52) U.S. Cl. CPC A61K 31/553; A61K 9/0014; A61K 9/0031; A61K 9/0034; A61K 9/006; A61K 9/06; A61K 9/08; A61K 9/107; A61K 47/10; A61K 47/12; A61K 47/14; A61K 47/183; A61K 47/22; A61K 47/24; A61K 47/26; A61K 47/32; A61K 47/36; A61P 17/00; A61P 17/02; A61P 17/06; A61P 17/14

(57) ABSTRACT

The object of the present invention is Udonitrectag (or Negermin or MTS. IUPAC name (1S,4R,5R,7S)-3,4-dibenzyl-2-oxo-6,8-dioxa-3-azabicyclo[3.2.1]octane-7-carboxylic acid lysine salt) and pharmaceutical formulation thereof for topical use for use in the treatment of dermatological diseases. i.e. of the skin and mucous membranes.

### FIELD OF THE INVENTION

The present invention relates to the field of pharmaceutical formulations for topical use for the treatment of irritated or damaged skin or mucous membranes diseases; in particular it relates to a formulation for topical use based on Udonitrectag [IUPAC Name: (1S,4R,5R,7S)-3,4-dibenzyl-2-oxo-6,8-dioxa-3-azabicyclo[3.2.1]octane-7-carboxylic acid lysine salt].

### STATE OF THE ART

Skin diseases affect men and women equally. The most common are: dermatitis (atopic, contact, seborrheic dermatitis), dermatosis and eczema; skin infections induced by bacteria and viruses (chlamydia, varicella zoster, papilloma virus, mycobacteria); skin infections induced by parasites (scabies); insect bites, diseases of the vaginal mucosa, burns, hemorrhoids, mycosis, acne, rosacea, psoriasis, benign and malignant tumors, erythema, itching, vitiligo, pigmentation disorders (seborrheic keratosis), warts, urticaria, alopecia, hair loss, follicular cysts, traumatic or surgical injuries, bedsores, and ulcers.

Furthermore, medicine is paying more and more attention to the role of “microcirculation” since its impairment seems to be the basis of many pathological processes. Microcirculation may be compromised by many factors, some of them are inexorable, such as aging, others are to be found among pathological states (such as diabetes, thyroid dysfunction, chronic renal failure), lifestyle and implications thereof. An example is the maintenance of constant hyperglycemia which increases the risk of vascular complications in large caliber vessels as well as in small capillaries, thus compromising microcirculation.

For this reason, and due to the importance of microcirculation in the supply of oxygen and nutrients to all the tissues of our body, it is clear how its chronic impairment may lead to serious diseases, such as diabetic foot. In fact, the reduction in the blood flow to the lower limbs favors the formation of skin ulcers with a poor tendency to heal which results, in the most serious cases, into diabetic feet. The latter makes the affected area more susceptible to infections which, if neglected, can lead to the need for limb amputation.

Diabetes is one of the fastest growing diseases worldwide. People with diabetes inevitably develop various complications, such as skin wounds, during their lifetime. Skin wounds, especially chronic ulcers, commonly develop in the feet of people with type 2 diabetes mellitus (T2DM), they are usually related to neuropathy (nerve damage), as well as arterial (blood vessel) diseases or trauma. Local treatment of chronic diabetic skin ulcer includes a number of different drugs, such as antibiotics, antimicrobials, wound disinfectants, creams, alginates, hydrocolloids, hydrogels, foams, and several specialized dressings in different combinations. However, at present, there are no commercially available drugs capable of effectively treating diabetic foot ulcers. This circumstance accounts for 85% of lower limb amputations of people with diabetes being preceded by foot ulcers that do not heal.

The technical problem that has not been solved yet in the state of the art is the continuous need for new and more efficient pharmaceutical remedies for the treatment of the above skin and mucous membrane diseases.

Neurotrophins (NGF and BDNF) are involved in skin physiology, as both keratinocytes and fibroblasts produce NGF, and epithelial cells express TrkA: the NGF receptor. Through interaction with the TrkA receptor, NGF exerts its effects in wound healing by regulating the activity of PI3K/Akt, ERK1/2, JNK and p38 MAPK. The activation of these biochemical pathways simultaneously leads to strong anti-apoptotic effects, and thus promotes survival and differentiation of epithelial and endothelial precursors, i.e. the cell types recruited to perform wound healing. However, the very poor pharmacokinetic properties of native neurotrophins essentially prevent their use as drugs for the treatment of skin diseases.

Udonitrectag (or Negermin or MT8, IUPAC name (1S,4R,5R,7S)-3,4-dibenzyl-2-oxo-6,8-dioxa-3-azabicyclo[3.2.1]octane-7-carboxylic acid lysine salt) is a non-peptide neurotrophins mimetic compound capable of binding both the TrkA receptor and the TrkB receptor. Udonitrectag has been tested in several in vitro and in vivo disease models in which apoptosis is the main process driving cell death (Scarpi et al. Cell Death and Disease 2012, 3, e339; doi: 10.1038/cddis.2012.80).

WO2004000324, in the name of the same Applicant, discloses 3-aza-bicyclo[3.2.1]octane derivatives as agonists of human neurotrophins, which are therefore useful for use in the treatment of diseases in which the functions of neurotrophins, in particular the functions of NGF, are implicated in a defective way: neurodegenerative disorders of the central nervous system, such as Alzheimer's disease (AD), amyotrophic lateral sclerosis (ALS), Huntington's disease, neuropathies, neural damage caused by hypoxia, ischemia, or trauma, inducing nerve cells apoptosis; acquired immunodeficiency diseases linked to reduced bioavailability of NGF, such as immunodeficiency of aging; diseases in which stimulation of neoangiogenesis is beneficial, such as myocardial infarction, stroke, wound healing or peripheral vascular disease; certain eye diseases, such as keratitis of various etiology, glaucoma, degenerative or inflammatory conditions of the retina.

WO2013140348, again in the name of the same Applicant, describes certain carboxylic acid derivatives of 3-aza-bicyclo[3.2.1]octane, among which MT8 which is (1S,4R,5R,7S)-3,4-dibenzyl-2-oxo-6,8-dioxa-3-azabicyclo[3.2.1]octane-7-carboxylic acid lysine salt (Udonitrectag), and medical use thereof, in particular in the treatment of all those disease related to ischemia-reperfusion, wherein ischemic conditions resulting from any reduction or block of blood flow are followed by subsequent re-establishment of oxygen/nutrient supply to the tissues, or for use in medical procedures involving ischemia-reperfusion.

WO/2021209910 describes some carboxylic acid derivatives of 3-aza-bicyclo[3.2.1]octane, among which Udonitrectag (MT8) (1S,4R,5R,7S)-3,4-dibenzyl-2-oxo-6,8-dioxa-3-azabicyclo[3.2.1]octane-7-carboxylic acid lysine salt, as activators of ADAR1 for use in the treatment of inflammatory disorders characterized by cytokine storm and/or uncontrolled immune response, such as SARS-COV-2. Udonitrectag has received “Orphan drug” designation for the treatment of neurotrophic keratitis and for therapeutic use in the field of solid organ transplantation.

### SUMMARY OF THE INVENTION

Object of the present invention is Udonitrectag (or Negermin or MT8, IUPAC name (1S,4R,5R,7S)-3,4-dibenzyl-2-oxo-6,8-dioxa-3-azabicyclo[3.2.1]octane-7-carboxylic acid lysine salt), and pharmaceutical formulation thereof, for topical use for use in the treatment of dermatological diseases, i.e. of the skin and mucous membranes.

The surprising efficacy of Udonitrectag in counteracting diabetes-induced endothelial cell dysfunction has been demonstrated through an in vitro study of endothelial dysfunction. Udonitrectag was also surprisingly successfully applied in an animal model of alopecia. Finally, with the topical administration of Udonitrectag it was unexpectedly possible to treat diabetic ulcers, traumatic wounds and psoriatic lesions which were incurable with treatments known in the state of the art.

### DETAILED DESCRIPTION OF THE INVENTION

Said diseases of the skin and mucous membranes (buccal, vaginal, anal mucous membranes) are preferably selected from the group consisting of vascular diseases (arteritis, phlebitis), dermatitis (atopic, contact, seborrheic dermatitis), dermatoses, eczema; skin infections induced by bacteria and viruses (chlamydia, varicella zoster, papilloma virus, mycobacteria); skin infections induced by parasites (scabies); mycosis, acne, rosacea, psoriasis, benign and malignant tumors, erythema, itching, vitiligo, pigmentation disorders (seborrheic keratosis), warts, urticaria, alopecia, hair loss, follicular cysts, insect bites, canker sores, diseases of the vaginal mucosa, hemorrhoids, anal fissures, burns, traumatic or surgical injuries, diabetic foot, bedsores and ulcers (diabetic or not).

The topical formulation according to the invention is in a semi-solid form or a solution selected from the group consisting of aqueous solutions, hydro-alcoholic solutions, gels, emulgels, lotions, sprayable lotions, spray solutions with propellants, O/W and W/O emulsions, ointments, sticks, ovules, suppositories, medicated plasters, medicated bandages, films, nanofibers, aspersion powders.

The topical formulation of the invention is suitable for both human and veterinary use.

The topical formulation of the invention preferably comprises Udonitrectag at 0.01-10% w/w, more preferably at 0.1-3%, even more preferably at 0.125-0.5%. Udonitrectag may be added into the composition as a crystalline or amorphous form.

The formulation preferably further comprises a chelating agent to ensure the stability of the active ingredient, the chelating agent being preferably EDTA or salts thereof, in a concentration of 0.1-0.2% w/w.

The composition preferably further comprises an absorption promoter, preferably hydrogenated phosphatidylcholine, in a concentration of 0.001-30% w/w.

The composition preferably further comprises hyaluronic acid in a concentration of 0.001-30% w/w. Hyaluronic acid sodium salt can be linear or crosslinked. Hyaluronic acid sodium salt preferably has a molecular weight of 300-3M kDa.

The composition of the invention includes suitable pharmaceutical grade excipients included in the “HANDBOOK OF PHARMACEUTICAL EXCIPIENTS” edited by Raymond C Rowe, Paul J Sheskey and Marian E Quinn, published by Pharmaceutical Press.

The excipients are preferably selected from the group consisting of solvents, co-solvents, preservatives, antioxidants, viscosifiers, pH adjuster, emulsifier.

The formulation preferably further comprises at least one solvent selected from the group consisting of purified water.

The formulation preferably further comprises at least one co-solvent selected from the group consisting of ethyl alcohol (in a concentration of 10-30% w/w), propylene glycol (in concentration of 3-7% w/w).

The formulation preferably further comprises at least one preservative, in a concentration of 0.01-0.3% w/w, selected from the group consisting of potassium sorbate, benzoic acid and 4-methyl-hydroxy-benzoate.

The formulation preferably further comprises a viscosifying agent, in a concentration of 0.5-1% w/w, selected from the group consisting of acrylic acid polymers crosslinked with allyl sucrose.

The formulation preferably further comprises at least one pH adjuster selected from the group consisting of 10% w/v NaOH solution.

The formulation preferably further comprises at least one emulsifier selected from the group consisting of non-ionic surfactants such as, for example, mixtures of mono-, di- and triglycerides and PEG-6 mono- and diesters of oleic acid (e.g. Labrafil®); capric and caprylic acid triglycerides (e.g. Labrafac®); mixtures of PEG-6 esters of stearic acid; ethylene glycol esters of palmitic and stearic acids and PEG-32 esters of stearic acid (e.g. Tefose®).

The formulation preferably further comprises at least one antioxidant selected from the group consisting of butylhydroxyanisole (BHA), alpha-tocopherol.

The present invention can be better understood in the light of the following embodiments.

### BRIEF DESCRIPTION OF THE FIGURES

FIGS. 1 and 2 show the effects of MT8 in a representative healing process in injured monolayers, in comparison with NGF or control medium (Example 16).

FIG.: 3 shows the effects of MT8 on the reduction of AGE-induced endothelial dysfunction in comparison with NGF+BDNF or control medium (Example 17)

FIGS. 4 and 5 show the comparative effects of a treatment of psoriatic lesions with a product of the state of the art and a composition according to the invention (Example 18)

FIGS. 6-16 show healing of severe skin lesions that were incurable with other treatments following topical application of Udonitrectag (Example 19-29).

FIG. 17 shows in vivo efficacy of Udonitrectag in an animal model of alopecia. (Example 30)

### EXPERIMENTAL PART

### Example No. 1: Udonitrectag-Based Gel at a Concentration of 2% w/w, for the Treatment of Diseases with Intact Skin

Batch 20210617-A

| | % w/w | ×60 g |
|---|---|---|
| Udonitrectag | 2.00 | 1.20 |
| Potassium sorbate | 0.2 | 0.12 |
| EDTA-disodium-dihydrate | 0.16 | 0.10 |
| Propylene Glycol | 5.00 | 3.00 |
| Carbopol ® 980 NF | 0.8 | 0.48 |
| 10% w/v NaOH solution | 4.13 | 2.48 |
| Ethyl alcohol | 20.0 | 12.0 |
| Purified water | q.s. to 100 | q.s. to 60 g |

Method for preparing a 60 g gel batch:
Phase 1 #. In the first step, 40.62 g of deionized water are placed in a Pyrex glass beaker and heated on a heating plate (F20500162, Velp, Italy) until a temperature of 80° C. is reached. Subsequently, 0.12 g of potassium sorbate, 1.20 g of Udonitrectag and 0.10 g of EDTA-disodium-dihydrate are added to it and kept under stirring (Rw 16, IKA, Germany), until complete dissolution.
Phase 2 #. The second phase is prepared by placing 3.00 g of propylene glycol and 0.48 g of Carbomer (Carbopol® 980 NF) in a Pyrex glass beaker. The two excipients are mixed until a dispersion is formed, so that Carbomer is finely suspended to avoid the formation of lumps.
Phase 3 #. The third step consists in adding the aqueous phase containing the active principle (Phase 1 #) to the suspension of Carbomer and propylene glycol (Phase 2 #). At this point, 12.00 g of ethyl alcohol are added to it, and the system is kept under slow stirring (Rw 16, IKA, Germany) (Speed=2) for one hour to allow for
Carbomer hydration, i.e. its complete dispersion in the liquid medium without incorporation of air bubbles.
Phase 4 #. The pH is checked with a pH-meter (Cyberscan pH 110, Eutech Instruments, USA) and the value adjusted with 2.48 g of a 10% w/v sodium hydroxide solution until a pH between 6 and 7 is reached.
Phase 5 #. Finally, the gel thus obtained is kept under slow stirring (Rw 16, IKA, Germany) (Speed=2) for one hour, and then placed in heralded aluminium tubes.

### Example No. 2: Udonitrectag-Based Gel at a Concentration of 0.5% w/w, for the Treatment of Vascular Diseases (Arteritis, Phlebitis)

Batch 20210720

| | % w/w | ×60 g |
|---|---|---|
| Udonitrectag | 0.5 | 0.3 |
| Potassium sorbate | 0.2 | 0.12 |
| EDTA-disodium-dihydrate | 0.16 | 0.10 |
| Propylene glycol | 5.00 | 3.00 |
| Carbopol ® 980 NF | 1.0 | 0.60 |
| 10% w/v NaOH solution | 4.75 | 2.85 |
| Ethyl alcohol | 20.0 | 12.0 |
| Purified water | q.s. to 100 | q.s. to 60 g |

Method for preparing a 60 g gel batch:
Phase 1 #. In the first step, 10.00 g of deionized water are placed in a Pyrex glass beaker together with 0.30 g of Udonitrectag in order to obtain an active ingredient solution.
Phase 2 #. In the second step, 31.03 g of deionized water are placed in a Pyrex glass beaker and heated on a heating plate (F20500162, Velp, Italy) until the temperature of 80° C. is reached. 0.12 g of potassium sorbate and 0.10 g of EDTA-disodium-dihydrate are added and kept under stirring (Rw 16, IKA, Germany), until complete dissolution.
Phase 3 #. In the third step, 12.0 g of ethyl alcohol, 3.00 g of propylene glycol and 0.60 g of Carbomer (Carbopol® 980 NF) are added. The mixture is kept under slow stirring (Rw 16, IKA, Germany) (Speed=2) for one hour to allow for Carbomer complete hydration.
Phase 4 #. The pH is checked with a pH-meter (Cyberscan pH 110, Eutech Instruments, USA) and the value adjusted with 2.85 g of a 10% w/v sodium hydroxide solution until a pH between 6 and 7 is reached.
Phase 5 #. The active ingredient solution (Phase #1) is combined with the aqueous solution forming Phase #2. The gel thus obtained is kept under slow stirring (Rw 16, IKA, Germany) (Speed=2) for one hour and then packaged in heralded aluminium tubes.

### Example No. 3: Udonitrectag-Based Emulgel at a Concentration of 2.0% w/w, for the Treatment of Diseases with Intact Skin and Microcirculation Diseases

In this formulation an absorption promoter (hydrogenated phosphatidylcholine) was used to increase Udonitrectag skin permeation.

Batch 20210604

| | % w/w | ×60 g |
|---|---|---|
| Udonitrectag | 2.00 | 1.20 |
| Potassium sorbate | 0.2 | 0.12 |
| EDTA-disodium-dihydrate | 0.16 | 0.10 |
| Phospholipon ® 80H (hydrogenated | 1.00 | 0.60 |
| phosphatidylcholine) | | |
| Propylene glycol | 5.00 | 3.00 |
| Carbopol ® 980 NF | 0.8 | 0.48 |
| 10% w/v NaOH solution | 2.42 | 1.45 |
| Ethyl alcohol | 20.0 | 12.0 |
| Purified water | q.s. to 100 | q.s. to 60 g |

Method for preparing a 60 g emulgel batch:
Phase 1 #. 41.05 g of deionized water are weighed in a Pyrex glass beaker and heated on a heating plate (F20500162, Velp, Italy) until a temperature of 80° C. is reached. 0.12 g of potassium sorbate, 1.20 g of Udonitrectag and 0.10 g of EDTA-disodium-dihydrate are added and kept under stirring (Rw 16, IKA, Germany), until complete dissolution.
Phase 2 #. 0.60 g of hydrogenated phosphatidylcholine (Phospholipon® 80H) are added to the solution obtained and the whole is placed under a homogenizer, at Speed 8 (Ultraturrax t25, IKA, Germany), for 10 minutes.
Phase 3 #. 12.00 g of ethyl alcohol, 3.00 g of propylene glycol and 0.48 g of Carbomer (Carbopol® 980 NF) are added to the homogenized suspension. The system is kept under slow stirring (Rw 16, IKA, Germany) (Speed=2) for one hour, in order to allow for Carbomer hydration.
The pH is checked with a pH-meter (Cyberscan pH 110, Eutech Instruments, USA) and the value adjusted with 1.45 g of a 10% w/v sodium hydroxide solution until a pH between 6 and 7 is reached.
Phase 5 #. Finally, the emulgel thus obtained is kept under slow stirring (Rw 16, IKA, Germany) for 10 minutes and, subsequently, packaged in heralded aluminium tubes.

### Example No. 4: Udonitrectag-Based Emulgel at a Concentration of 0.5% w/w. for the Treatment of Bedsores and Diabetic Foot

In this formulation an absorption promoter (hydrogenated phosphatidylcholine) was used to increase Udonitrectag skin permeation and hyaluronic acid (sodium salt) to promote wound healing.

Batch 20210617-B

| | % w/w | ×60 g |
|---|---|---|
| Udonitrectag | 0.5 | 0.30 |
| Potassium sorbate | 0.2 | 0.12 |
| EDTA-disodium-dihydrate | 0.16 | 0.10 |
| Hyaluronic acid, sodium salt | 1.00 | 0.60 |
| (M.W. 1.2 MDa) | | |
| Phospholipon ® 80H (hydrogenated | 1.00 | 0.60 |
| phosphatidylcholine) | | |
| Carbopol ® 980 NF | 0.4 | 0.24 |
| 10% w/v NaOH solution | 1.65 | 0.99 |
| Purified water | q.s. to 100 | q.s. to 60 g |

Method for preparing a 60 g emulgel batch:
Phase 1 #. 10.00 g of deionized water are weighed and placed in a Pyrex glass beaker in which 0.30 g of Udonitrectag are dissolved. The solution is left aside.
Phase 2 #. 23.53 g of deionized water are weighed and placed in a Pyrex glass beaker and then heated on a heating plate (F20500162, Velp, Italy) until a temperature of 80° C. is reached. 0.12 g of potassium sorbate, 0.60 g of hyaluronic acid 1.2 MDa and 0.10 g of EDTA-disodium-dihydrate are added and stirred (Rw 16, IKA, Germany), until complete dissolution.
0.60 g of hydrogenated phosphatidylcholine (Phospholipon® 80H) are added to the solution obtained, which is placed under a homogenizer, at Speed 8 (Ultraturrax t25, IKA, Germany), for 10 minutes.

Phase 3 #. Separately, the third phase of the production process is prepared, in which 23.52 g of deionized water and 0.24 g of Carbomer (Carbopol® 980 NF) are added into a Pyrex glass beaker. The system is kept under slow stirring (Rw 16, IKA, Germany) (Speed=2), for one hour to allow for Carbomer hydration.

Phase 4 #. The pH is checked with a pH-meter (Cyberscan pH 110, Eutech Instruments, USA) and the pH value, which should be between 6 and 7, is adjusted with 0.99 g of a 10% w/v sodium hydroxide solution.

Phase 5 #. Finally, the solution in which the Udonitrectag was dissolved (Phase 1 #) and the suspension containing hyaluronic acid (Phase 2 #) are combined with the Carbomer-containing solution (Phase 3 #).

The emulgel thus obtained is kept under slow stirring (Rw 16, IKA, Germany) for 10 minutes and then packaged in heralded aluminium tubes.

### Example No. 5: Udonitrectag-Based Emulgel at a Concentration of 0.5% w/w. for the Treatment of Diseases with Intact Skin

In this formulation an absorption promoter (hydrogenated phosphatidylcholine) was used to increase Udonitrectag skin permeation.

Batch 20210720

| | % w/w | ×60 g |
|---|---|---|
| Udonitrectag | 0.50 | 0.30 |
| Potassium sorbate | 0.20 | 0.12 |
| EDTA-disodium-dihydrate | 0.16 | 0.10 |
| Phospholipon ® 90H | 1.00 | 0.60 |
| (hydrogenated phosphatidylcholine) | | |
| Propylene glycol | 5.00 | 3.00 |
| Carbopol ® 980 NF | 1.0 | 0.60 |
| 10% w/v NaOH solution | 4.50 | 2.70 |
| Ethyl alcohol | 20.0 | 12.0 |
| Purified water | q.s. to 100 | q.s. to 60 g |

Method for preparing a 60 g emulgel batch:
Phase 1 #. 40.58 g of deionized water are weighed, then placed in a Pyrex glass beaker and heated on a heating plate (F20500162, Velp, Italy) until a temperature of 80° C. is reached. 0.12 g of potassium sorbate, 0.30 g of Udonitrectag and 0.10 g of EDTA-disodium-dihydrate are added and kept under stirring (Rw 16, IKA, Germany) until complete dissolution.
Phase 2 #. 0.60 g of hydrogenated phosphatidylcholine (Phospholipon® 90H) are added to the solution obtained which is placed under a homogenizer, at Speed 8 (Ultraturrax t25, IKA, Germany), for 10 minutes.
Phase 3 #. 12.00 g of ethyl alcohol, 3.00 g of propylene glycol and 0.60 g of Carbomer (Carbopol® 980 NF) are added to the homogenized suspension. The system is kept under slow stirring (Rw 16, IKA, Germany) (Speed=2) for one hour, in order to allow for complete Carbomer hydration.
Phase 4 #. The pH is checked with a pH-meter (Cyberscan pH 110, Eutech Instruments, USA) and the value adjusted with 2.70 g of a 10% w/v sodium hydroxide solution until a pH between 6 and 7 is reached.
Phase 5 #. Finally, the emulgel thus obtained is kept under slow stirring (Rw 16, IKA, Germany) for 10 minutes and then packaged in heralded aluminium tubes.

### Example No. 6: Udonitrectag-Based Emulgel at a Concentration of 0.5% w/w, for the Treatment of Bedsores and Diabetic Foot

In this formulation, a highly purified absorption promoter (90% hydrogenated phosphatidylcholine) was used to increase Udonitrectag skin permeation and hyaluronic acid (sodium salt) to promote wound healing.

Batch 20210722

| | % w/w | ×60 g |
|---|---|---|
| Udonitrectag | 0.50 | 0.30 |
| Potassium sorbate | 0.20 | 0.12 |
| EDTA-disodium-dihydrate | 0.16 | 0.10 |
| Phospholipon ® 90H | 1.00 | 0.60 |
| (hydrogenated phosphatidylcholine) | | |
| Hyaluronic acid 1.0 MDa | 1.00 | 0.60 |
| Carbopol ® 980 NF | 0.60 | 0.36 |
| 10% w/v NaOH solution | 2.67 | 1.60 |
| Purified water | q.s. to 100 | q.s. to 60 g |

Method for preparing a 60 g emulgel batch:
Phase 1 #. 10.00 g of deionized water are weighed and placed in a Pyrex glass beaker in which 0.30 g of Udonitrectag are added and dissolved, under gentle stirring.
Phase 2 #. Separately, 23.16 g of deionized water are heated on a heating plate (F20500162, Velp, Italy) until a temperature of 80° C. is reached. 0.12 g of potassium sorbate, 0.60 g of hyaluronic acid 1.0 MDa and 0.10 g of EDTA-disodium-dihydrate are added and kept under stirring (Rw 16, IKA, Germany), until complete dissolution. Then, 0.60 g of hydrogenated phosphatidylcholine (Phospholipon® 90H) are added to the solution obtained, which is homogenized at Speed 8 (Ultraturrax t25, IKA, Germany), for 10 minutes.
Phase 3 #. Separately, 23.16 g of deionized water are weighed in a Pyrex glass beaker to which 0.36 g of Carbomer (Carbopol® 980 NF) are added. The system is kept under slow stirring (Rw 16, IKA, Germany) (Speed=2) for one hour, in order to allow for Carbomer hydration.
Phase 4 #. The pH is checked with a pH-meter (Cyberscan pH 110, Eutech Instruments, USA) and the value adjusted with 1.60 g of a 10% w/v sodium hydroxide solution until a pH between 6 and 7 is reached.
Phase 5 #. Finally, the solution in which the Udonitrectag was dissolved (Phase 1 #) and the suspension containing hyaluronic acid (Phase 2 #) are combined with the Carbomer containing solution (Phase 3 #).
The emulgel thus obtained is kept under slow stirring (Rw 16, IKA, Germany) for 20 minutes; then packaged in heralded aluminium tubes.

### Example No. 7: Udonitrectag-Based O/W Emulsion at a Concentration of 2% w/w for the Treatment of Inflammatory Skin Diseases (Psoriasis)

Batch 20210603

| | % w/w | ×60 g |
|---|---|---|
| Udonitrectag | 2.00 | 1.20 |
| Tefose ® 63 | 20.00 | 12.00 |
| Labrafil ® MC 1944 CS | 4.00 | 2.40 |
| Labrafac ® CC | 6.00 | 3.60 |
| Phospholipon ® 80H | 3.00 | 1.80 |
| Benzoic acid | 0.2 | 0.12 |
| 10% w/v NaOH solution | 1.67 | 1.00 |
| Purified water | q.s. to 100 | q.s. to 60 g |

Method for preparing a 60 g emulsion batch:
Phase 1 #. Preparation of the fat phase. All the lipids present in the formulation are added into a Pyrex glass beaker:
12.00 g of a polyoxyl-6-stearate type I, ethylene glycol stearate and polyoxyl-32-stearate type I (Tefose® 63) mixture
2.40 g of Oleoyl-macrogol-6 glycerides (Labrafil® M 1944 CS)
3.60 g of Propylene glycol dicaprylate/dicaprate (Labrapac® CC)
1.80 g of hydrogenated phosphatidylcholine (Phospholipon® 80H).
Phase 2 #. The beaker is then placed on a heating plate (F20500162, Velp, Italy), heated and kept at 75° C. until the lipids are fully melted.
Phase 3 #. Separately, the aqueous phase is prepared, in which 37.88 g of deionized water, 1.20 g of Udonitrectag, 0.12 g of benzoic acid and 1.00 g of solution at 10% w/v sodium hydroxide are placed in a Pyrex glass beaker. The beaker is then heated and stirred with a magnet at a temperature of 75° C. (F20500162, Velp, Italy), until complete dissolution.
Phase 4 #. Finally, in the fourth step, an emulsion is formed, in which both the aqueous and lipid phases should be kept at the same temperature. The phase inversion method is used, in which the aqueous phase containing Udonitrectag is added to the fat phase containing the lipids. Once the two phases are combined, they are homogenized, with a homogenizer (Ultraturrax t25, IKA, Germany), at Speed 8, for 10 minutes.
Phase 5 #. The emulsion formed is allowed to cool until a temperature of 25° C. is reached, under slow stirring (Speed=2) (Rw 16, IKA, Germany), in order to allow its structuring without incorporation of air bubbles. The emulsion is then packaged in heralded aluminium tubes.

### Example No. 8: Udonitrectag-Based O/W Emulsion at a Concentration of 0.5% w/w for the Treatment of Inflammatory Skin Diseases (Dermatitis, Eczema)

Batch 20210719-A

| | % w/w | ×60 g |
|---|---|---|
| Udonitrectag | 0.5 | 0.3 |
| Tefose ® 63 | 22.00 | 13.20 |
| Labrafil ® MC 1944 CS | 4.00 | 2.40 |
| Labrafac ® CC | 6.00 | 3.60 |
| Phospholipon ® 90H | 3.00 | 1.80 |
| Alpha-tocopherol acetate | 0.2 | 0.12 |
| Potassium sorbate | 0.2 | 0.12 |
| 10% w/v NaOH solution | 0.07 | 0.04 |
| Purified water | q.s. to 100 | q.s. to 60 g |

Method for preparing a 60 g O/W emulsion batch:
Phase 1 #. Preparation of the fat phase. All the lipids present in the formulation are added into a Pyrex glass beaker:
13.20 g of polyoxyl-6-stearate type I, ethylene glycol stearate and polyoxyl-32-stearate type I (Tefose® 63) mixture 2.40 g of Oleoyl-macrogol-6 glycerides (Labrafil® M 1944 CS)
3.60 g of Propylene glycol dicaprylate/dicaprate (Labrapac® CC)
1.80 g of hydrogenated phosphatidylcholine (Phospholipon® 90H)
0.12 g of alpha-tocopherol acetate

The beaker is then placed on a heating plate (F20500162, Velp, Italy), heated and kept at 75° C. until the lipids are fully melted.
Phase 2 #. Separately, 10.00 g of deionized water are weighed and 0.30 g of Udonitrectag are added and dissolved into it.
Phase 3 #. Separately, the aqueous phase is prepared, wherein 0.12 g of potassium sorbate, 0.04 g of 10% w/v sodium hydroxide solution and 28.42 g of water deionized are placed into a Pyrex glass beaker; the beaker is heated and stirred with a magnet at a temperature of 75° C. (F20500162, Velp, Italy), until complete dissolution.
Phase 4 #. Emulsification step: both the aqueous and lipid phases should be kept at the same temperature. The phase inversion method is used for emulsion formation. These phases are homogenized, with a homogenizer (Ultraturrax t25, IKA, Germany), at Speed 8, for 10 minutes.
Phase 5 #. The emulsion formed is allowed to cool until a temperature of 25° C. is reached, under slow stirring (Speed=2) (Rw 16, IKA, Germany), in order to allow its structuring without incorporation of air bubbles. Once the temperature indicated above is reached, the active ingredient solution is added to the continuous phase. The emulsion is then kept under slow stirring (Rw 16, IKA, Germany) (Speed=2) for half an hour.
The cream is then packaged in heralded aluminium tubes.

### Example No. 9: Udonitrectag-Based O/W Emulsion at 0.5% w/w Concentration for the Treatment of Inflammatory Skin Diseases (Dermatitis, Eczema)

Batch 20210719-B

| | % w/w | ×60 g |
|---|---|---|
| Udonitrectag | 0.5 | 0.3 |
| Tefose ® 63 | 22.00 | 13.20 |
| Labrafil ® MC 1944 CS | 4.00 | 2.40 |
| Labrafac ® CC | 6.00 | 3.60 |
| Phospholipon ® 90H | 3.00 | 1.80 |
| BHA | 0.02 | 0.01 |
| Potassium sorbate | 0.2 | 0.12 |
| 10% w/v NaOH solution | 0.08 | 0.05 |
| Purified water | q.s. to 100 | q.s. to 60 g |

Method for preparing a 60 g O/W emulsion batch:
Phase 1 #. Preparation of the fat phase. All the lipids present in the formulation are added into a Pyrex glass beaker:
13.20 g of polyoxyl-6-stearate type I, ethylene glycol stearate and polyoxyl-32-stearate type I (Tefose® 63) mixture
2.40 g of Oleoyl-macrogol-6 glycerides (Labrafil® M 1944 CS)
3.60 g of Propylene glycol dicaprylate/dicaprate (Labrapac® CC)
1.80 g of hydrogenated lecithin (Phospholipon® 90H)
0.01 g of Butyl-hydroxy anisole (BHA)
The beaker is then placed on a heating plate (F20500162, Velp, Italy), heated and kept at 75° C. until the lipids are fully melted.
Phase 2 #. Separately, 10.00 g of deionized water are weighed and 0.30 g of Udonitrectag are added and dissolved into it.
Phase 3 #. In a third beaker, the aqueous phase is prepared, in which 28.42 g of deionized water, 0.12 g of potassium sorbate, 0.04 g of 10% w/v solution of sodium hydroxide are weighed. The beaker is heated and kept under magnetic stirring at a temperature of 75° C. (F20500162, Velp, Italy), until complete dissolution of the substances.
Phase 4 #. Emulsification step: Both Phase1 # and 3 #(aqueous and lipid phases), should be kept at the same temperature. The phase inversion method is used for emulsion formation. These phases are homogenized, with a homogenizer (Ultraturrax t25, IKA, Germany), at Speed 8, for 10 minutes.
Phase 5 #. The emulsion formed is allowed to cool until the temperature of 25° C. is reached, under slow stirring (Speed=2) (Rw 16, IKA, Germany). Once the temperature indicated above is reached, the active ingredient solution (Phase 2 #) is added to the emulsion. The emulsion is then kept under slow stirring (Rw 16, IKA, Germany) (Speed=2) for half an hour.
The cream is then packaged in heralded aluminium tubes.
Among the pharmaceutical forms for cutaneous use, Udonitrectag can be usefully employed in trichological lotions for the treatment of Alopecia.
A formulation example is given below.

### Example No. 10: Udonitrectag-Based Trichological Lotion at a Concentration of 2.0% w/w for the Treatment of Alopecia

Batch 20210604-A

| | % w/w | ×60 g |
|---|---|---|
| Udonitrectag | 2.0 | 1.20 |
| Carbopol ® 980NF | 0.05 | 0.03 |
| 1% w/v NaOH solution | 0.92 | 0.55 |
| Potassium sorbate | 0.2 | 0.12 |
| Phospholipon ® 80H | 1.00 | 0.6 |
| Labrafil ® M 1944 CS | 5.00 | 3.00 |
| Ethyl alcohol | 20.00 | 12.00 |
| Purified water | q.s. to 100 | q.s. to 60 g |

Method for preparing a 60 g lotion batch:
Phase 1 #. In a Pyrex glass beaker, 42.50 g of deionized water are weighed and heated up to 80° C. on a heating plate (F20500162, Velp, Italy). 0.12 g of potassium sorbate and 1.20 g of Udonitrectag are added to the Phase and kept under stirring (Rw 16, IKA, Germany), until complete dissolution.
Phase 2 #. 0.60 g of hydrogenated phosphatidylcholine (Phospholipon® 80H) are added to the solution obtained; then it is homogenized using Speed 8 (Ultraturrax t25, IKA, Germany), for 10 minutes. Phase 2 # is cooled to room temperature.
Phase 3 #. 12.00 g of ethyl alcohol and 0.03 g of Carbomer (Carbopol® 980 NF) are added to the homogenized suspension; the system is kept under slow stirring (Rw 16, IKA, Germany) (Speed=2) for one hour to allow for Carbomer complete hydration.
Phase 4 #. 3.00 g of Oleoyl-macrogol-6 glycerides (Labrafil® M 1944 CS) are added to Phase 3 # and kept under slow stirring (Rw 16, IKA, Germany) (Speed=2) for 10 minutes.
Phase 5 #. The pH is checked using a pH-meter (Cyberscan pH 110, Eutech Instruments, USA) and the value is adjusted with 0.55 g of a 1% w/v sodium hydroxide solution until a pH between 6 and 7 is reached.
Phase 6 #. The lotion thus obtained is then sonicated in an ultrasonic bath (mod. 450, Branson, USA) for 30 minutes and, subsequently, kept under slow stirring (Rw 16, IKA, Germany) (Speed=2) for 10 minutes. The lotion is packaged in white transparent glass bottles equipped with a spray pump.
The various Udonitrectag-containing pharmaceutical forms for cutaneous use can also be used in the treatment of skin ulcers in pets or large animals.
A formulation example of a formulation for veterinary use is provided below.

### Example No. 11: Udonitrectag-Based Lotion for Veterinary Use_at_0.5% w/w Concentration for the Treatment of Skin Ulcers

| | Batch 20210728 | |
|---|---|---|
| | % w/w | ×60 g |
| Udonitrectag | 0.50 | 0.30 |
| Hyaluronic acid, 1.00 MDa, sodium salt | 0.20 | 0.12 |
| 1% w/v NaOH solution | 3.03 | 1.82 |
| Potassium sorbate | 0.20 | 0.12 |
| Phospholipon ® 90H | 1.00 | 0.60 |
| EDTA-disodium-dihydrate | 0.16 | 0.10 |
| Carbopol ® 980NF | 0.05 | 0.03 |
| Labrafil ® M 1944 CS | 1.00 | 0.60 |
| Purified water | q.s. to 100 | q.s. to 60 g |

Method of preparing a 60 g lotion batch for veterinary use:
Phase 1 #. 10.0 g of deionized water and 0.3 g of Udonitrectag are weighed in a Pyrex glass beaker and kept under gentle stirring until the active ingredient is dissolved.
Phase 2 #. Separately, 46.32 g of deionized water are added to a Pyrex glass beaker and heated up to 80° C. on a heating plate (F20500162, Velp, Italy). 0.12 g of potassium sorbate, 0.10 g of EDTA-disodium-dihydrate and 0.12 g of hyaluronic acid sodium salt 1.00 MDa are added and kept under stirring (Rw 16, IKA, Germany), until complete dissolution.
Once the hyaluronic acid is uniformly dispersed in the solution, 0.60 g of hydrogenated phosphatidylcholine (Phospholipon® 90H) are added to it and homogenized at Speed 8 (Ultraturrax t25, IKA, Germany), for 10 minutes.
Phase 3 #. 0.03 g of Carbomer (Carbopol® 980 NF) are added to the homogenized suspension; the system is kept under slow stirring (Rw 16, IKA, Germany) (Speed=2) for about an hour to allow for Carbomer hydration.
Phase 4 #. 0.60 g of Oleoyl-macrogol-6 glycerides (Labrafil® M 1944 CS) are added to Phase 3 # and kept under slow stirring (Rw 16, IKA, Germany) (Speed=2) for 10 minutes.
Phase 5 #. The pH is checked using a pH-meter (Cyberscan pH 110, Eutech Instruments, USA) and the value adjusted with 1.82 g of a 1% w/v sodium hydroxide solution until the pH value is between 6 and 7.
Phase 6 #. Finally, the lotion thus obtained is sonicated in an ultrasonic bath (mod. 450, Branson, USA) for 30 minutes and kept under slow stirring (Rw 16, IKA, Germany) (Speed=2) for 10 minutes.
The lotion is packaged in white transparent glass bottles equipped with a spray pump.

### Example No. 12: Udonitrectag-Based Gel, for Vaginal Use, at 0.5% w/w Concentration, for the Treatment of the Vaginal Mucosa

Batch 20211001

| | % w/w | ×60 g |
|---|---|---|
| Udonitrectag | 0.5 | 0.3 |
| Potassium sorbate | 0.2 | 0.12 |
| 1% w/v NaOH solution | 0.92 | 0.55 |
| Hyaluronic acid, 1 MDa sodium salt | 0.5 | 0.3 |
| Purified water | q.s. to 100 | q.s. to 60 g |

Method of preparing a 60 g gel batch:
Phase 1 #. 48.73 g of deionized water are placed in a Pyrex glass beaker and heated on a heating plate (F20500162, Velp, Italy) until a temperature of 80° C. is reached. Subsequently, 0.12 g of potassium sorbate and 0.5 g of hyaluronic acid sodium salt are added to it and kept under stirring (Rw 16, IKA, Germany), until complete dissolution.
Phase 2 #. Separately, 10.00 g of deionized water and 0.3 g of Udonitrectag are weighed in a Pyrex glass beaker, while stirring until a clear solution is obtained.
Phase 3 #. The aqueous phase containing hyaluronic acid sodium salt (Phase 1 #) is combined with the active ingredient aqueous solution (Phase 2 #). The system is kept under slow stirring (Rw 16, IKA, Germany) (Speed=2) for one hour.
Phase 4 #. The pH is checked with a pH-meter (Cyberscan pH 110, Eutech Instruments, USA) and the value adjusted with 0.55 g of a 1% w/v sodium hydroxide solution until a pH between 6 and 7 is reached.
Phase 5 #. The gel thus obtained is kept under slow stirring (Rw 16, IKA, Germany) (Speed=2) for one hour.

The gel is packaged in heralded aluminium tubes together with a vaginal applicator.

### Example No. 13: Udonitrectag-Based Enema, for Rectal Use, at 2.0% w/w Concentration, for the Treatment of Anal Fissures

Batch 20211004

| | % w/w | ×60 g |
|---|---|---|
| Udonitrectag | 2.0 | 1.20 |
| Potassium sorbate | 0.2 | 0.12 |
| 1% w/v NaOH solution | 0.92 | 0.55 |
| Crosslinked hyaluronic acid | 0.5 | 0.3 |
| Phospholipon ® 90H | 0.3 | 0.18 |
| Purified water | q.s. to 100 | q.s. to 60 g |

Method of preparing a 60 g enema batch:
Phase 1 #. 10.00 g of deionized water are weighed and placed in a Pyrex glass beaker in which 1.20 g of Udonitrectag are dissolved.
Phase 2 #. Separately, 47.65 g of deionized water are weighed, placed in a Pyrex glass beaker and heated on a heating plate (F20500162, Velp, Italy) until a temperature of 80° C. is reached. 0.12 g of potassium sorbate and 0.30 g of hyaluronic acid sodium salt are added to it and kept under stirring (Rw 16, IKA, Germany), until complete dissolution.
0.18 g of hydrogenated phosphatidylcholine (Phospholipon® 90H) are added to the obtained solution, and homogenized with a homogenizer (Ultraturrax t25, IKA, Germany), at Speed 8, for 10 minutes.
Phase 3 #. The pH is checked with a pH-meter (Cyberscan pH 110, Eutech Instruments, USA) and the value is adjusted with 0.55 g of a 1% w/v sodium hydroxide solution until a pH between 6 and 7 is reached.
Phase 4 #. Phase 2 # is combined with Phase 1 # and the enema is kept under slow stirring (Rw 16, IKA, Germany) for 20 minutes.
The enema is packaged in rectal applicators at the volume of 20 g/dose.

### Example No. 14: Udonitrectag-Based Gel, for Buccal Use, at 0.5% w/w Concentration, for the Treatment of Canker Sores

Batch 20211005

| | % w/w | ×60 g |
|---|---|---|
| Udonitrectag | 0.5 | 1.20 |
| Potassium sorbate | 0.2 | 0.12 |
| Methyl 4-hydroxybenzoate | 0.18 | 0.11 |
| 1 MDa Hyaluronic acid | 1.5 | 0.9 |
| Phospholipon ® 90H | 0.3 | 0.18 |
| 1% w/v NaOH solution | 0.90 | 0.54 |
| Purified water | q.s. to 100 | q.s. to 60 g |

Method of preparing a 60 g gel batch:
Phase 1 #. 10.00 g of deionized water are weighed and placed in a Pyrex glass beaker in which 0.3 g of Udonitrectag is dissolved.
Phase 2 #. Separately, 47.79 g of deionized water are weighed, placed in a Pyrex glass beaker, and heated on a heating plate (F20500162, Velp, Italy) until reaching a temperature of 80° C. 0.12 g of potassium sorbate, 0.11 g of methyl 4-hydroxybenzoate and 0.9 g of hyaluronic acid sodium salt are added to it and dissolved under stirring (Rw 16, IKA, Germany).
0.18 g of hydrogenated phosphatidylcholine (Phospholipon® 90H) are added to the solution obtained which is homogenized using a homogenizer (Ultraturrax t25, IKA, Germany) at Speed 8, for 10 minutes.
Phase 3 #. The pH is checked with a pH-meter (Cyberscan pH 110, Eutech Instruments, USA) and the value adjusted with 0.54 g of a 1% w/v sodium hydroxide solution until a pH between 6 and 7 is reached.
Phase 4 #. The Udonitrectag solution (Phase 1 #) is combined with the solution of Phase 2 #, then the system is kept under slow stirring (Rw 16, IKA, Germany) for 20 minutes. The gel thus obtained is packaged in heralded aluminium tubes.

### Example No. 15-Permeation Studies

The purpose of using absorption promoters in semi-solid formulations is to implement the drug permeation rate and, consequently, drug absorption through the skin/mucosa.

To demonstrate this function of uptake promoters, a topical gel formulation containing 2% Udonitrectag (Batch 20210617-B, see Ex. 4) was analyzed in terms of IVPT and compared with the same formulation (batch 20210604, see Ex. 3) into which 1% hydrogenated phosphatidylcholine was included (as absorption promoter).

The skin permeation studies were carried out using Franz cells with porcine skin in accordance with the monograph USP 37<1724> SEMISOLID DRUG PRODUCTS —PERFORMANCE TESTS

The flow expressed as J (μg/cm²/h) was calculated for these formulations. Below are the values obtained from the IVPT:
Batch 20210617 (Ex. 1: gel containing 2% Udonitrectag-without absorption promoter):

$$J ( μg / cm 2 / h ) = 9.81$$

Batch 20210604 (Ex. 3: emulgel containing 2% Udonitrectag—with absorption promoter such as hydrogenated phosphatidylcholine at 1% w/w)

$$J ( μg / cm 2 / h ) = 20.59$$

The results show how an absorption promoter, such as for example hydrogenated phosphatidylcholine, increases the permeability of the active ingredient contained in the formulation and, consequently, its skin absorption.

### Example No. 16—In Vitro Study on Wound Healing

To evaluate whether MT8 is able to induce faster healing of injured human keratinocytes (HACAT cell line) monolayers, an in vitro “wound healing” model was used. HACAT cells were seeded in 6-well culture dishes and grown to 90%-95% confluency. A standardized wound was created by scratching the HACAT cell monolayer with a sterile micropipette tip under an inverted microscope. Then, the cells were washed with PBS and treated with 50 μM MT8 or 4 nM NGF, and images of the wound healing process were recorded at different time points.

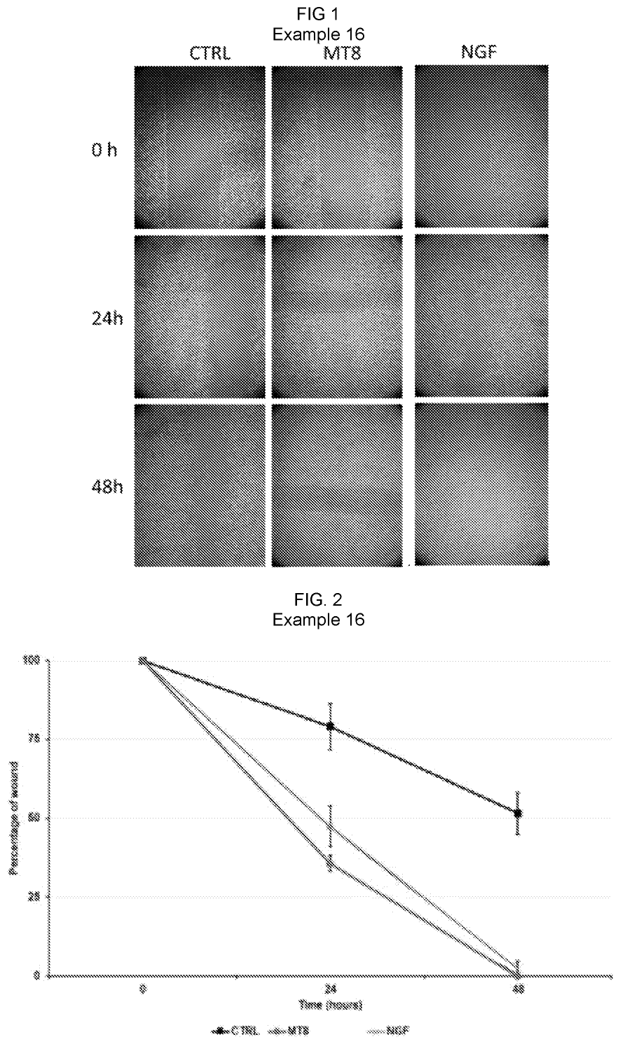

FIG. 1 shows a representative healing process in wounded monolayers treated with MT8, NGF, or control medium. FIG. 2 shows the wound distances as a function of time; it is apparent that MT8 induces a faster repair process, as compared to cultures treated with control medium.

### Example No. 17—In Vitro Study on the Reduction of Endothelial Dysfunction

Endothelial dysfunction is one of the key determinants in the development of diabetes-related diseases. One of the main mechanisms of development of these complications is the formation of advanced glycation end products (AGEs). These AGEs accumulate in long-lived tissue proteins, causing crosslinking and developing inflammation and thickening of basal membranes. This leads to the development of complications such as retinopathy, neuropathy, nephropathy, and atherosclerosis. The activity of MT8 in counteracting diabetes-induced endothelial cell dysfunction was demonstrated through the in vitro study on endothelial dysfunction.

BSA-AGE and ECM-AGE were produced by Maillard reaction and Amadori rearrangement and used to evaluate oxidative stress and apoptosis process induced by AGEs in endothelial cell cultures.

To evaluate whether MT8 is able to counteract AGE-induced endothelial cell dysfunction, Human Umbilical Vein Endothelial Cells (HUVECs) were seeded on ECM-AGE-coated plates and incubated with complete growth medium, in the presence or absence of 20 UM MT8, or 2 nM NGF+2 nM BDNF for 4 days. The cells were then lysed with 100 UL of CellTiter Glo, which allows for ATP release from the cells. The ATP content (directly proportional to cell viability) was measured by luciferase assay.

The results showed that both AGE products (soluble and ECM) generated are able to induce apoptosis in endothelial cells. FIG. 3 shows that, similarly to NGF+BDNF, MT8 is able to maintain endothelial cell viability by preventing AGE-induced metabolic derangement.

### Example No. 18—Treatment of Psoriasis

A patient diagnosed with psoriasis having lesions on the calves of both legs underwent the following treatment:

| Day | Right leg | Left leg |
|---|---|---|
| 1-15 | FAGRON, morning and | Udonitrectag, 0.5% gel- |
| | evening | cream, and then |
| | | FAGRON, morning and |
| | | evening |
| 16-20 | FAGRON, evening | Udonitrectag, 0.5% gel- |
| | | cream, and then |
| | | FAGRON, evening |
| 21-30 | Udonitrectag, 0.5% gel- | Udonitrectag, 0.5% gel- |
| | cream, morning and | cream, morning and |
| | evening | evening |

FAGRON: galenic preparation (0.05% clobetasol propionate, 10% urea in occluvan q.s. to 100%)

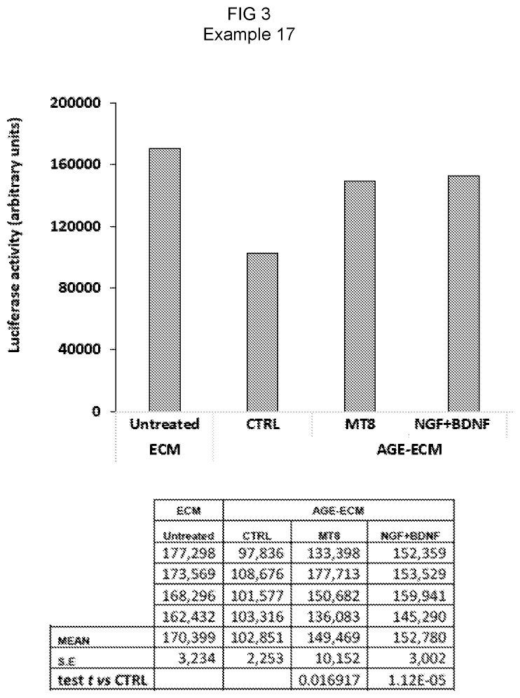

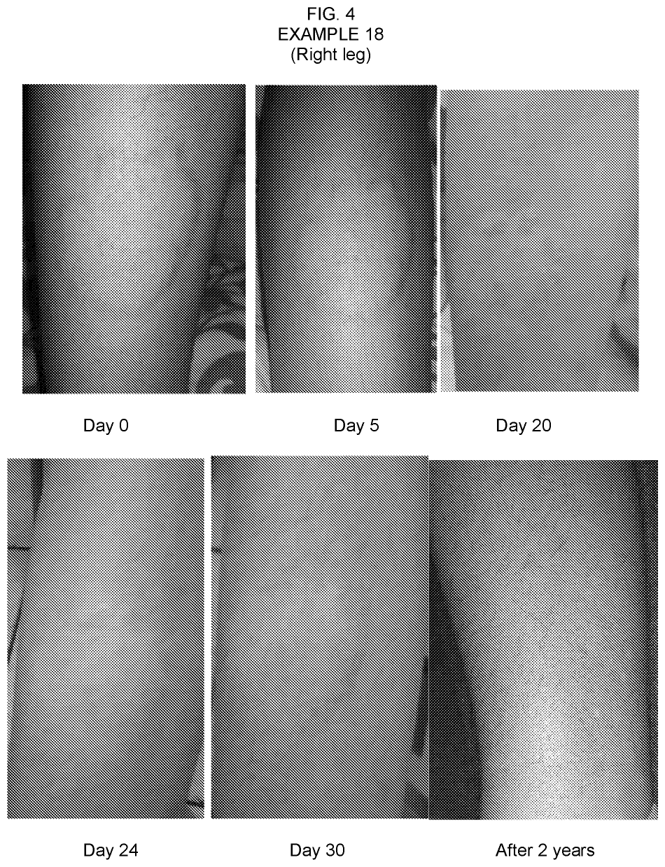

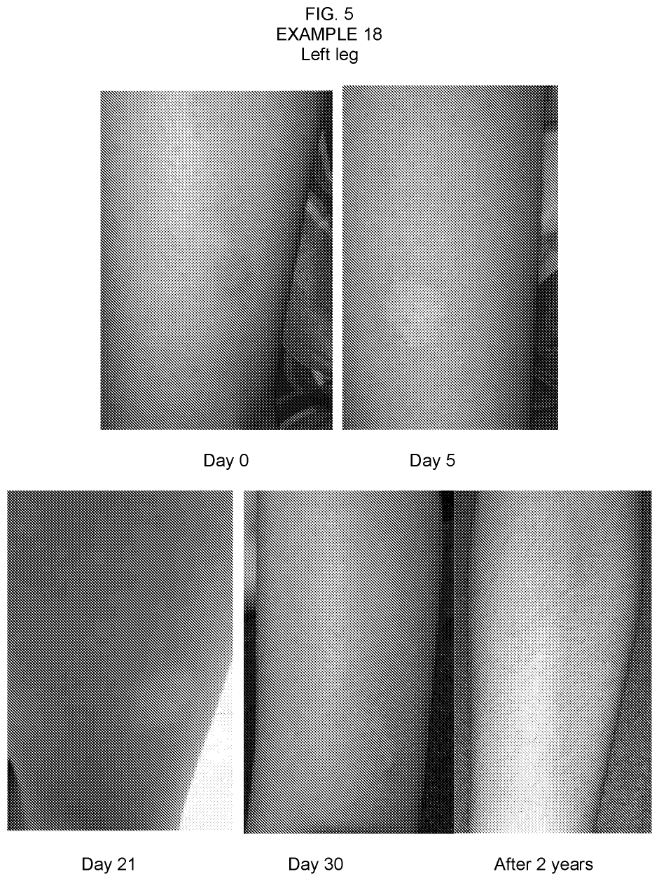

The treatment with FAGRON did not produce any improvement, while the product with Udonitrectag produced visible improvements in the lesions. Furthermore, psoriasis was completely healed on both the left and right leg, as revealed by skin examination two years after the end of the treatment (see FIGS. 4-5).

Examples 19-29: All the patients reported in these examples were treated with a Udonitrectag gel-cream having a similar composition to what reported in Example 4.

### Example No. 19

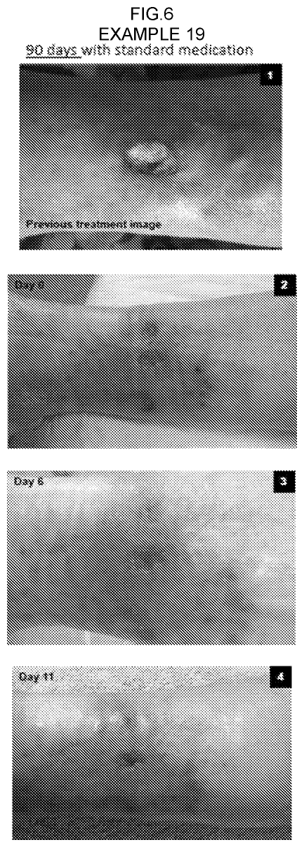

PATIENTS Q.I. Age: 86 Gender: Female-Non-smoker, BMI 24.5
Concomitant diseases: micro/macrovascular alterations, recurrent infections, allergies to silver, hyaluronic acid, hydrocolloid, and honey.
Medical treatment. Inoperable antibiotic multi-resistance. Pulmonary neoplasm, herpes zoster on the left shoulder involving the entire left upper limb, peripheral venous vascular disease, deforming arthritis.
Site: above the inner malleolus of the right leg, plus additional satellite lesions of the main treatment.
Type: Venous vascular injury occurred in May 2018; she self-medicated at home until October 2018, then she was assisted by the wound care team with unsatisfactory results.
Previous treatment: Previous 12 months of home medication with 10% povidone iodine without any significant clinical improvement.
Characteristics on Day 0: Granulation tissue is present throughout the lesion and epithelium reconstitution fails. The jagged edges of the lesion are erythematous and infected. The periwound skin is erythematous and edematous, with erythema extending to the malleolar area. The lesion is lightly exuding, with foul-smelling, serous exudate. Contaminated/infected wound. When changing the dressing, the pain expressed is equal to 8 (VAS pain scale); there is still discomfort and sometimes pain even when the dressing is not changed. Complex wound due to comorbidities.
Treatment with Udonitrectag: Wound cleaning performed with sterile saline solution (0.9% sodium chloride) and Microdacyn, followed by Urgotul and finally 1% Udonitrectag gel-cream, covered with a light dressing; 2 dressing changes per week for a total of 5 dressings with 1% Udonitrectag gel-cream. Clinical improvement apparent from the first application of 1% Udonitrectag gel-cream. Clear and continuous reparative growth until almost complete re-epithelialization. See FIG. 6.

### Example No. 20

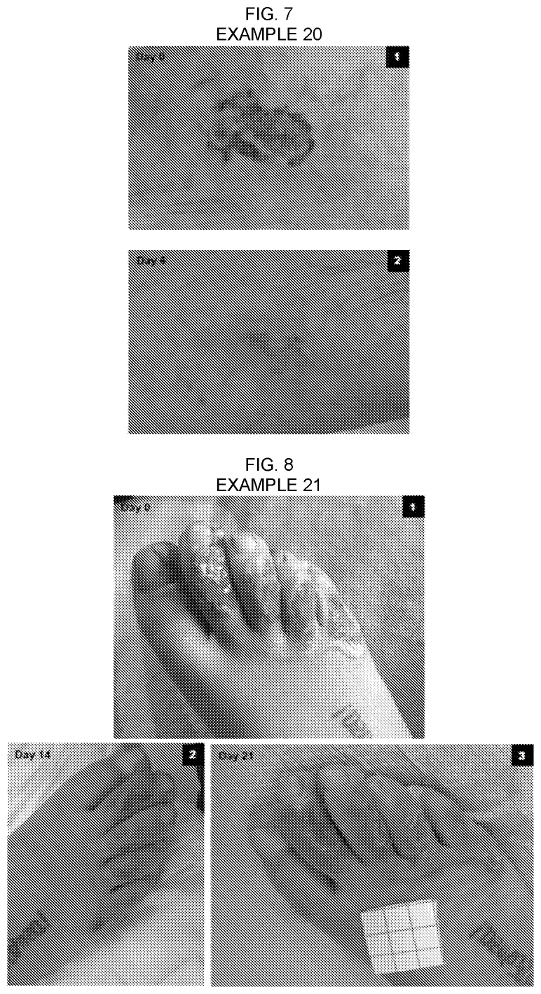

PATIENTS: D.L. Age: 77 years Gender: Male, BMI 26.2 (slightly overweight), non-smoker, but history as a heavy smoker (40 cigarettes a day), laryngectomized for non-metastatic laryngeal cancer.
Concomitant diseases: Hypertension, diabetes.
Site: Right forearm
Type: Extravasation due to antibiotic therapy (ciproxin)
Characteristics on Day 0: Lesion with presence of granulation tissue, jagged edematous margins, presence of necrosis at the margins. Mild purulent exudate.
Pain only on dressing change with expressed pain equal to 6 (VAS Pain Scale).
Prior Treatment: No prior treatment
Treatment with Udonitrectag gel-cream: Wound cleansing with 0.9% sodium chloride, applied 1% Udonitrectag gel-cream, followed by sticky polyurethane foam dressing; 3 dressing changes per week for a total of 2 dressings with 1% Udonitrectag gel-cream.
Dramatic reduction of edema at first dressing change. Rapid reparative growth at second dressing change. Re-epithelialization at third dressing change. See FIG. 7.

### Example No. 21

PATIENTS: Y.T. Age: 26 Gender: female, smoker
Concomitant diseases: None.
Site: Right foot
Type: Wound from traumatic injury in a young and healthy individual.
Previous treatment: Home dressing for 1 month (31 days) with hydrogen peroxide, gauze, and TNT (non-woven fabric) dressing
Characteristics on Day 0: Wounds from extensive traumatic injury to right foot toes. Udonitrectag gel-cream treatment: Wound cleansing with sterile 0.9% sodium chloride saline solution, debridement at each visit, Microdacyn for 10 minutes, 1% Udonitrectag gel-cream, followed by Urgotul Ag, sterile gauze, light ankle bandage and Class 1 compression stockings. 2 dressing changes per week for a total of 8 dressings with 1% Udonitrectag gel-cream. See FIG. 8.

### Example No. 22

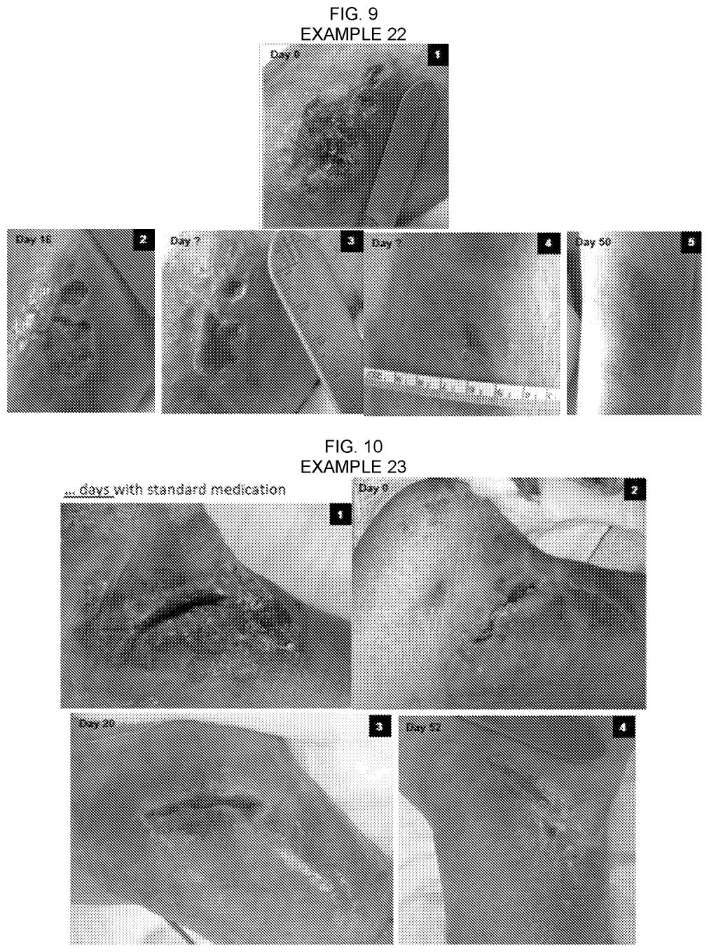

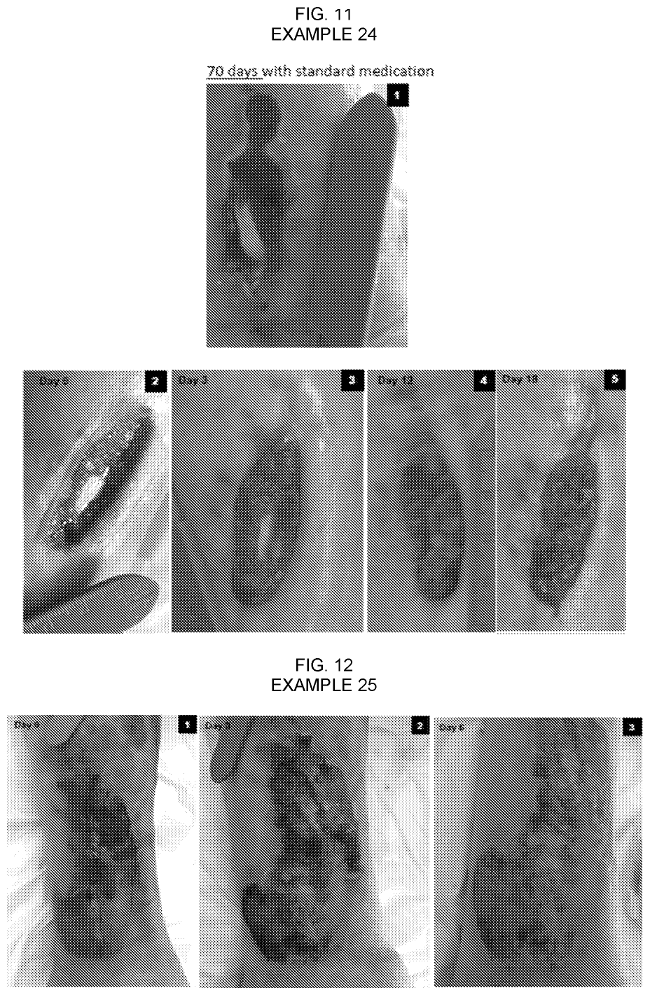

PATIENTS: M.F. Age: 49. Gender: Male
Concomitant diseases: Cardiovascular diseases, severe vascular disease and microvasculature impairment.
Site: Left leg
Type: Traumatic injury.
Characteristics on Day 0: Very difficult wound to heal due to severe vascular disease and compromised microvasculature. Clear capillary and skin fragility.
Prior Treatment: No prior treatment.
Udonitrectag gel-cream treatment: Wound cleansing with sterile saline (0.9% sodium chloride), Microdacyn wound cleanser, then 1% Udonitrectag gel-cream, followed by Urgotul and Biatain, dressing and elastic compression. See FIG. 9.

### Example No. 23

PATIENTS G.M. Age: 57 Gender: Male
Concomitant diseases: None.
Site: Right ankle.
Type: Surgical wound dehiscence.
Characteristics on Day 0: Surgical wound dehiscence.
Previous treatment: Surgical treatment, 10% povidone iodine and Aquacel AG dressing.
Treatment with Udonitrectag gel-cream: wound cleansing with sterile saline solution (0.9% sodium chloride), Microdacyn wound cleanser, 1% Udonitrectag gel-cream, followed by dressing with Urgotul and TNT; 2 dressing changes per week for a total of 26 dressings with 1% Udonitrectag gel-cream. See FIG. 10.

### Example No. 24

PATIENTS R.M. Age: 51 Gender: Male
Concomitant diseases: None.
Site: Right central leg.
Type: Surgical wound dehiscence.
Characteristics on Day 0: Surgical wound dehiscence
Previous treatment: dressing with sterile gauze and TNT.
Treatment with Udonitrectag gel-cream: wound cleansing with saline solution (0.9% sodium chloride) and Microdacyn wound cleanser for 10 minutes, 1% Udonitrectag gel-cream, followed by Urgotul AG, sterile gauze and light bandage; 3 dressing changes per week for a total of 6 dressings with 1% Udonitrectag gel-cream. See FIG. 1.

### Example No. 25

PATIENTS G.P. Age: 89 years Gender: female
Concomitant diseases: Vascular diseases, skin fragility in the elderly, post-fall femoral fracture.
Site: Left leg.
Type: Lesion injury, post-fall femur fracture.
Characteristics on Day 0: Lesion wound in very elderly patient with severe skin fragility and very slow wound healing rate.
Prior Treatment: No prior treatment.
Treatment with Udonitrectag gel-cream: Wound cleansing with sterile saline (0.9% sodium chloride), debridement at each visit, 1% Udonitrectag gel-cream, followed by tacky polyurethane foam and light dressing; 3 dressing changes per week for a total of 2 dressings with 1% Udonitrectag gel-cream. See FIG. 12.

### Example No. 26

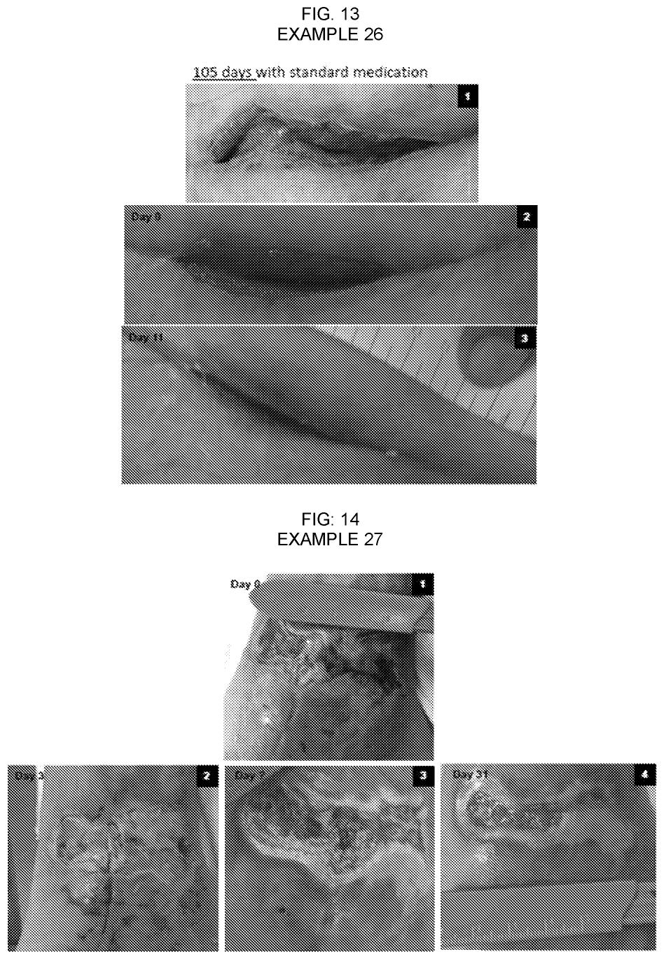

PATIENTS R.R. Age: 81 Gender: Female
Concomitant diseases: Abdominal hernia, hypertension, weight loss.
Site: Abdominal wall.
Type: Surgical wound dehiscence.
Characteristics on Day 0: Surgical wound in very elderly patient with severe skin fragility and very slow wound healing rate.
Previous treatment: Vacuum-assisted closure (VAC) therapy for 180 days Udonitrectag gel-cream treatment: Wound cleansing with sterile saline (0.9% sodium chloride), Microdacyn wound cleanser, 1% Udonitrectag gel-cream, followed by Urgotul and TNT dressing; 2 dressing changes per week for a total of 5 dressings with 1% Udonitrectag gel-cream. See FIG. 13.

### Example No. 27

PATIENTS G.F. Age: 42 Gender: Male
Concomitant diseases: None.
Site: Right ankle.
Type: Surgical wound dehiscence.
Characteristics on Day 0: Surgical wound dehiscence
Previous treatment: Surgical treatment due to wound necrosis and wound dehiscence.
Treatment with Udonitrectag: Wound cleansing with sterile saline (0.9% sodium chloride), wound cleanser, Microdacyn wound cleanser, % Udonitrectag gel-cream, followed by dressing with Urgotul and TNT; 2 dressing changes per week for a total of 16 dressings with 1% Udonitrectag gel-cream. See FIG. 14.

### Example No. 28

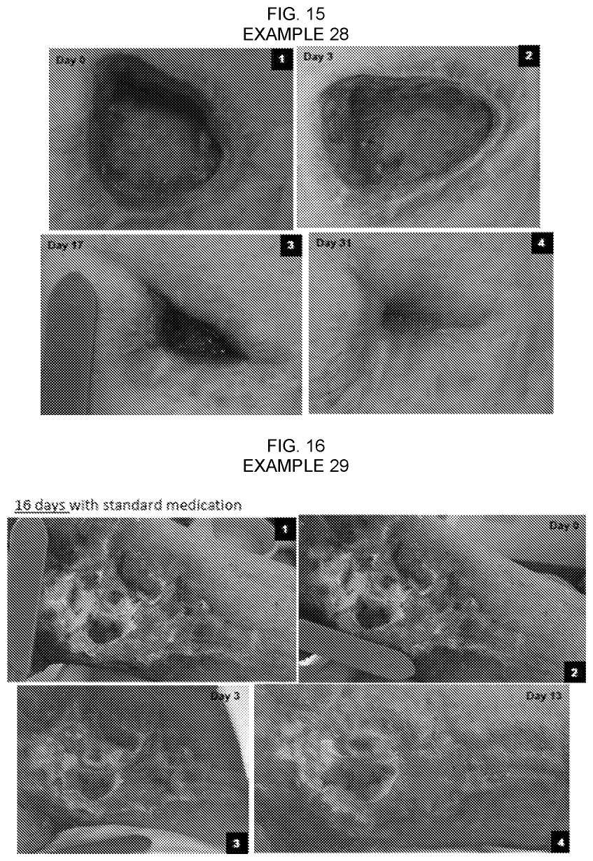

PATIENTS L.B. Age: 50 years Gender: Male
Concomitant diseases: Perforating diverticulitis.
Site: Abdominal wall.
Type: Surgical wound dehiscence.
Characteristics on Day 0: Surgical wound dehiscence
Previous treatment: None.
Udonitrectag treatment: Wound cleansing with sterile saline (0.9% sodium chloride) Microdacyn wound cleanser for 10 minutes, Udonitrectag gel-cream (1%), followed by Urgotul and Biatain super; 2 dressing changes per week for a total of 16 dressings with 1% Udonitrectag gel-cream. See FIG. 15.

### Example No. 29

PATIENTS E.B. Age: 45 Gender: female
Concomitant disorders: Severe obesity (BMI 49.1).
Site: Inner malleolus of the right leg.
Type: Traumatic road injury, multiple broken fractures, lacerated and bruised right ankle wound with exposed bone in an African-American subject.
Characteristics on Day 0: Lacerated wound
Previous treatment: Surgical treatment.
Treatment with Udonitrectag gel-cream: Wound cleansing with sterile saline (0.9% sodium chloride), Microdacyn wound cleanser, 1% Udonitrectag gel-cream, followed by Urgotul and TNT dressing; 2 dressing changes per week for a total of 7 dressings with 1% Udonitrectag gel-cream. See FIG. 16.

### Example No. 30-In Vivo Study in an Animal Model of Androgenetic Alopecia

Udonitrectag was studied in an in vivo model of androgenetic alopecia.

For this purpose, male C57BL/6 mice were shaved with an animal clipper at 8 weeks of age, when all follicles are in the telogen phase. After depilation, 4 mg of DHT (dissolved in 100 μL of ethanol) were injected subcutaneously into the back of each animal. The mice were then divided into two groups: vehicle-treated and treated with Udonitrectag 100 μM in PBS. Udonitrectag 100 μM solution, or vehicle (PBS only) were applied topically twice a day for 2 weeks and hair growth was recorded at the beginning and at the end of treatment.

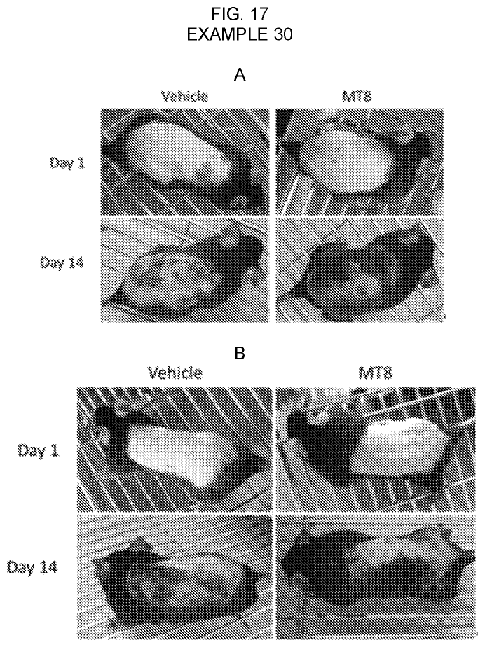

The results obtained (FIG. 17) showed that the treatment with Udonitrectag 100 μM induces hair regrowth much faster and more intensively than vehicle treatment.

1-10. (canceled)

11. A method for the topical treatment of a dermatological disease, said method comprising administering topically to a subject in need thereof a therapeutically effective amount of Udonitrectag.

12. The method according to claim 11, wherein said disease is selected from the group consisting of vascular disease, dermatitis and dermatosis, eczema, skin infection induced by bacteria and viruses, skin infection induced by parasites; mycosis, acne, rosacea, psoriasis, benign or malignant tumor, erythema, itching, vitiligo, pigmentation disorder, wart, urticaria, alopecia, hair loss, follicular cysts, insect bite, canker sores, disease of the vaginal mucosa, hemorrhoids, anal fissures, burn, traumatic or surgical injurie, diabetic foot, bedsore, and ulcer.

13. The method according to claim 11, wherein the subject in need is a human or veterinary subject.

14. A topical pharmaceutical formulation comprising Udonitrectag at 0.01-10% w/w, and at least one other pharmaceutically acceptable ingredient.

15. The formulation according to claim 14 in semi-solid form or solution selected from the group consisting of aqueous solutions, hydro-alcoholic solutions, gels, emulgels, lotions, sprayable lotions, spray solutions with propellants, O/W and A/W emulsions, ointments, sticks, ovules, suppositories, medicated plasters, medicated bandages, films, nanofibers, aspersion powders.

16. The formulation according to claim 14, further comprising a chelating agent to ensure the stability of the active ingredient.

17. The formulation according to claim 16, wherein the chetaling agent is EDTA or a salt thereof at a concentration of 0.1-0.2% wt.

18. The formulation according to claim 14, further comprising hyaluronic acid at a concentration of 0.001-30% w/w.

19. The formulation according to claim 14, further comprising an absorption promoter.

20. The formulation according to claim 19, wherein the absorption promoter is hydrogenated phosphatidylcholine, at a concentration of 0.001-30% w/w.

21. The formulation according to claim 14, wherein the at least one other pharmaceutically acceptable ingredient is selected from the group consisting of solvent, co-solvents, preservatives, antioxidants, viscosizers, pH adjusters, emulsifiers.

22. A method for the topical treatment of a dermatological disease, said method comprising administering topically to a subject in need thereof a therapeutic effective amount of a formulation according to claim 14.

23. The method according to claim 22, wherein said disease is selected from the group consisting of vascular disease, dermatitis and dermatosis, eczema, skin infection induced by bacteria and viruses, skin infection induced by parasites; mycosis, acne, rosacea, psoriasis, benign or malignant tumor, erythema, itching, vitiligo, pigmentation disorder, wart, urticaria, alopecia, hair loss, follicular cysts, insect bite, canker sores, disease of the vaginal mucosa, hemorrhoids, anal fissures, burn, traumatic or surgical injurie, diabetic foot, bedsore, and ulcer.

24. The method according to claim 22, wherein the subject in need is a human or a veterinary subject.
